(19) United States
(12) Patent Application Publication
(10) Pub. No.: US 2007/0264322 A1
(43) Pub. Date: Nov. 15, 2007

(54) Method for making liposomes conjugated with temperature-sensitive ligands

(76) Inventors: Ken Shi-Kun Huang, Castro Valley, CA (US); Zengji Li, San Ramon, CA (US); Jinkang Wang, San Francisco, CA (US); Guoyang Zhang, San Jose, CA (US); John Grigsby, Berkeley, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/801,713

(22) Filed: May 9, 2007

Related U.S. Application Data
(60) Provisional application No. 60/799,422, filed on May 10, 2006.

(51) Int. Cl. A61K 31/704; A61K 9/127

(52) U.S. Cl. 424/450; 977/907; 514/34

(57) ABSTRACT

The present invention relates to a method of making a liposome composition. In particular, the invention relates to a method of making liposomes targeted to a specific cell receptor for delivery of a liposome-entrapped drug to the cell. In one embodiment, the process involves the incorporation of lipid-linkers to the surface of pre-formed liposomes, preferably at a higher temperature, followed by the conjugation of one or more temperature-sensitive ligands to the linkers associated with the liposome surface at a lower temperature to avoid deactivation of the temperature sensitive ligands. The present invention also is directed to a product prepared according to the foregoing process, and its use to treat subjects. The present invention is also directed to a kit containing lipid-linker, ligand and pre-formed liposome.

### CROSS-REFERENCE TO RELATED APPLICATIONS

This application claims the benefit of U.S. Provisional Application No. 60/799,422, filed May 10, 2006, incorporated herein by reference in its entirety.

### FIELD

The present invention relates to a method of making a liposome composition. In particular, the invention relates to a method of making liposomes targeted to a specific cell receptor for delivery of a liposome-entrapped drug to the cell. In one embodiment, the process involves the incorporation of lipid-linkers to the surface of pre-formed liposomes, preferably at a higher temperature, followed by the conjugation of one or more temperature-sensitive ligands to the lipid-linkers associated with the liposome surface at a lower temperature to avoid deactivation of the temperature-sensitive ligands. The present invention is also directed to a product prepared according to the foregoing process, and its use to treat subjects. The present invention is also directed to a kit containing lipid-linker, ligand and pre-formed liposome.

### BACKGROUND

Liposomes are spherical vesicles comprised of concentrically ordered lipid bilayers that encapsulate an aqueous phase. Liposomes serve as a delivery vehicle for therapeutic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, so-called Stealth® or long-circulating liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Targeted-liposomes have targeting-ligands or affinity moieties associated or attached to the surface of the liposomes. The targeting-ligands may be antibodies or fragments thereof, in which case the liposomes are referred to as immunoliposomes. When administered systemically, targeted-liposomes deliver the entrapped therapeutic agent to a target tissue, region or, cell. Because targeted-liposomes are directed to a specific region or cell, healthy tissue is not exposed to the therapeutic agent. Such targeting-ligands can be attached directly to the liposomes' surfaces by covalent coupling of the targeting-ligand to the polar head group residues of liposomal lipid components (see, for example, U.S. Pat. No. 5,013,556). This approach, however, is suitable primarily for liposomes that lack surface-bound polymer chains, as the polymer chains interfere with interaction between the targeting-ligand and its intended target (Klibanov, A. L., et al., Biochim. Biophys. Acta., 1062:142-148 (1991); Hansen, C. B., et al., Biochim. Biophys. Acta, 1239:133-144 (1995)).

Alternatively, the targeting ligands can be attached to the free ends of the polymer chains forming the surface coat on the liposomes (Allen. T. M., et al., Biochim. Biophys. Acta, 1237:99-108 (1995); Blume, G. et al., Biochim. Biophys. Acta, 1149:180-184 (1993)). In this approach, the targeting-ligand is exposed and readily available for interaction with the intended target.

Various approaches have been described for preparing liposomes having a targeting-ligand attached to the distal end of liposome-attached polymer chains. One conventional approach involves preparation of lipid vesicles which include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting-ligand after liposome formation. In another conventional approach, the lipid-polymer-ligand conjugate is included in the lipid composition at the time of liposome formation (see, for example, U.S. Pat. Nos. 6,224,903, 5,620,689). In a more recent approach, commonly referred to as the “Insertion Method”, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973, 6,316,024, 6,210,707). The incorporation is due to incubation of the suspension of the conjugate with the pre-formed liposomes at a higher temperature (in one embodiment 50-60° C.) that increases the incorporation efficiency.

While liposomes carrying an entrapped agent and bearing surface-bound targeting-ligands, i.e., targeted, therapeutic liposomes, are prepared by any of these approaches, the preferred method of preparation is the Insertion Method, where pre-formed liposomes are incubated with the targeting conjugate to achieve insertion of the targeting conjugate into the liposomal bilayers. In this approach, liposomes are prepared by a variety of techniques, such as those detailed in Szoka, F., Jr., et al., Ann. Rev. Biophys. Bioeng., 9:467 (1980), and specific examples of liposomes prepared in support of the present invention will be described below. Typically, the liposomes are multilamellar vesicles (MLVs), which can be formed by simple lipid-film hydration techniques. In this procedure, a mixture of liposome-forming lipids dissolved in a suitable organic solvent is evaporated in a vessel to form a thin film, which is then covered by an aqueous medium. The lipid film hydrates to form MLVs, typically with sizes between about 0.1 to 10 microns.

The liposomes can include a vesicle-forming lipid derivatized with a hydrophilic polymer to form a surface coating of hydrophilic polymer chains on the liposomes' surface. Addition of a lipid-polymer conjugate is optional, since after the insertion step, the liposomes will include lipid-polymer-targeting-ligand. Additional polymer chains added to the lipid mixture at the time of liposome formation and in the form of a lipid-polymer conjugate result in polymer chains extending from both the inner and outer surfaces of the liposomal lipid bilayers. Addition of a lipid-polymer conjugate at the time of liposome formation is typically achieved by including between 1-20 mole percent of the polymer-derivatized lipid with the remaining liposome forming components, e.g., vesicle-forming lipids. Exemplary methods of preparing polymer-derivatized lipids and of forming polymer-coated liposomes have been described in U.S. Pat. Nos. 5,013,556, 5,631,018 and 5,395,619, which are incorporated herein by reference. It will be appreciated that the hydrophilic polymer may be stably coupled to the lipid, or coupled through an unstable linkage, which allows the coated liposomes to shed the coating of polymer chains as they circulate in the bloodstream or in response to a stimulus.

The liposomes also include a therapeutic or diagnostic agent, and exemplary agents are provided below. The selected agent is incorporated into liposomes by standard methods, including (i) passive entrapment of a water-soluble compound by hydrating a lipid film with an aqueous solution of the agent, (ii) passive entrapment of a lipophilic compound by hydrating a lipid film containing the agent, and (iii) loading an ionizable drug against an inside/outside liposome pH gradient. Other methods, such as reverse-phase evaporation, are also suitable.

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS—MANUFACTURING AND PRODUCTION TECHNOLOGY, P. Tyle, Ed., Marcel Dekker, New York, pp. 267-316 (1990)).

After formation of the liposomes, a targeting-ligand is incorporated to achieve a cell-targeted, therapeutic liposome. The targeting-ligand is incorporated by incubating the pre-formed liposomes with the lipid-polymer-ligand conjugate, prepared as described above. The pre-formed liposomes and the conjugate are incubated under conditions effective to achieve association with the conjugate and the liposomes, which may include interaction of the conjugate with the outer liposome bilayer or insertion of the conjugate into the liposome bilayer. More specifically, the two components are incubated together under conditions which achieve associate of the conjugate with the liposomes in such a way that the targeting-ligand is oriented outwardly from the liposome surface, and therefore available for interaction with its cognate receptor. It will be appreciated that the conditions effective to achieve such association or insertion are determined based on several variables, including, the desired rate of insertion, where a higher incubation temperature may achieve a faster rate of insertion, the temperature to which the ligand can be safely heated without affecting its activity, and to a lesser degree the phase transition temperature of the lipids and the lipid composition. It will also be appreciated that insertion can be varied by the presence of solvents, such as amphipathic solvents including polyethyleneglycol and ethanol, or detergents.

The targeting conjugate, in the form of a lipid-polymer-ligand conjugate, will typically form a solution of micelles when the conjugate is mixed with an aqueous solvent. The micellar solution of the conjugates is mixed with a suspension of pre-formed liposomes for incubation and association of the conjugate with the liposomes or insertion of the conjugate into the liposomal lipid bilayers. The incubation is effective to achieve associate or insertion of the lipid-polymer-antibody conjugate with the outer bilayer leaflet of the liposomes, to form an immunoliposome.

Despite the success of the conventional methods of conjugating targeting-ligands to liposomes and the Insertion Method, there are several drawbacks. Conventional methods of conjugating targeting-ligands to liposomes are complex and take a lot of time, generally on the order of 4-5 hours. Further, it is difficult to maintain the activity of lipid-polymer conjugates during the entire liposome preparation process until the targeting-ligand is conjugated. The Insertion Method is simpler and less expensive, but is problematic because it is performed in unfavorable conditions (i.e., high pH, higher temperatures, long incubation times). Elevated temperatures are a problem because many targeting-ligands are temperature sensitive and are likely to lose biological activity during the insertion process at high temperatures. Further, the Insertion Method is expensive, since the efficiency by which the lipid-polymer-ligand conjugates are inserted into the pre-formed liposomes is not as acceptable as one would desire, and results in uneven distribution of the lipid-polymer-ligand conjugates on the surface of the liposome due to the lipid lateral diffusion after insertion.

A need, therefore, exists for a method of making liposomes with targeting-ligands that would not be adversely affected by high pH and high temperature conditions and long incubation times. A need also exists, for a method of making liposomes that are simple, inexpensive and result in even distribution of lipid-polymer-ligand conjugates on the surface of liposomes.

### SUMMARY OF THE INVENTION

The present invention provides a novel method for preparing liposomes targeted to a specific cell receptor for delivery of a liposome-entrapped drug to the cell. In one embodiment, lipid-linkers for ligand conjugation are inserted into a pre-formed liposome at higher temperatures, and then ligands are conjugated to the lipid-linkers covalently on the surface of the pre-formed liposome at a lower temperature. In another embodiment, the lipid-linkers are combined with the pre-formed liposomes at temperatures in the range from about 50-70 degrees C., and then ligands are conjugated to the lipid-linkers covalently on the surface of the pre-formed liposome at room temperature.

Another aspect of the present invention is directed to a product prepared according to the foregoing process, and its use to treat subjects for a disease or disorder.

In another aspect of the present invention, the present invention is directed to a kit containing lipid linker, ligand and pre-formed liposome.

The method of the present invention combines advantages of previously established methods for preparing targeting-liposomes, while overcoming the disadvantages of these prior methods. A primary advantage is that lipid-linkers are inserted to the pre-formed liposomes rather than ligand-lipid conjugates. Since there is no conjugated ligands in the method of the present invention, the process can be carried on in a relative harsh condition (lower pH, higher temperature and longer incubation time) to improve the efficiency of lipid-linker incorporation into the membrane of liposome surface. Therefore, ligand conjugation to the liposomes can take place at low temperature to avoid deactivation of some temperature-sensitive ligands, such as, antibody, antibody fragment Fab′, single chain Fv and other temperature-sensitive protein or peptide ligands.

Another primary advantage is that the cost of the process is reduced. Since insertion efficiency is always lower than conjugation efficiency, and lipid-ligand conjugates are always more expensive than lipid-linkers, an excess amount of lipid-linkers can be used to insert into the liposome, rather than using an excess amount of lipid-polymer-ligand conjugates in order to achieve the same number of ligands per liposome ratio. Further, since limited number of linker-lipids distribute on the liposome surface evenly due to the lipid lateral diffusion after insertion, the chances of different lipid-linkers conjugating to different sites of the same protein ligand, such as, Fab′, might be reduced significantly compared to the chances of multiple linker-lipids conjugating to one Fab′ by using a conventional Mal-PEG-DSPE micelle incubation process in the Insertion Method.

These and other objects and features of the invention will be more fully appreciated when the following detailed description of the invention is read in conjunction with the accompanying drawings.

### BRIEF DESCRIPTION OF THE DRAWINGS

FIG. 1A-B are microscopic images of A375-S2 Human Melanoma cell internalization of an anti-integrin antibody Fab′-conjugated liposome and a liposome without the Fab′ conjugation.

FIG. 2A-B are charts of the in vitro cytotoxicity of A375-S2 Human Melanoma cells treated by anti-integrin antibody Fab′-conjugated liposomes with doxorubicin at 4° C. and liposomes without the Fab′ conjugation with doxorubicin at 4° C.

FIG. 3A-B are charts of the in vitro cytotoxicity of A375-S2 Human Melanoma cells treated by anti-integrin antibody Fab′-conjugated liposomes with doxorubicin at 37° C. and liposomes without the Fab′ conjugation with doxorubicin at 37° C.

### DETAILED DESCRIPTION OF THE INVENTION

I. Definitions

Unless otherwise noted, the term “vesicle-forming lipid” refers to any lipid capable of forming part of a stable micelle or liposome composition and typically including one or two hydrophobic, hydrocarbon chains or a steroid group and may contain a chemically reactive group, such as an amine, acid, ester, aldehyde or alcohol, at its polar head group.

As used herein, a “lipid-linker” is a vesicle-forming lipid derivatized on at least one end with a hydrophilic polymer to form a surface coating of hydrophilic polymer chains on the liposomes' surface when they are incorporated into the liposome. In one embodiment, the end-functionalized lipid-polymer derivative is one described above where the free polymer end is reactive or “activated” (see, for example, U.S. Pat. Nos. 6,326,353 and 6,132,763). In one embodiment, the activated conjugate is included in the liposome composition and the activated polymer ends are reacted with a targeting-ligand after the lipid-linker is inserted into the liposome. In one embodiment, the lipid-linker is inserted into the liposome at a temperature that is higher than the temperature at which a ligand is later attached to the lipid-linker. In another embodiment, the lipid-linker is a “lipopolymer” as that term is defined herein.

As used herein, an “antibody” includes whole antibodies and any antigen binding fragment or single chain fragment thereof. Thus the antibody includes any protein or peptide containing molecule that comprises at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian growth factor receptor. Examples of binding fragments encompassed within the term “antigen binding portion” of an antibody include (i) a Fab fragment, a monovalent fragment consisting of the VL, VH, CL and CH, domains; (ii) a F(ab′)₂ fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., Nature, 341 :544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science, 242:423-426 (1988), Huston et al., Proc. Natl. Acad. Sci. USA, 85:5879-5883 (1988)). Such single chain antibodies are also intended to be encompassed within the term antibody and with “antigen-binding portion” of an antibody. These antibody fragments are obtained using conventional techniques known to those with skill in the art, and the fragments are screened for utility in the same manner as are intact antibodies.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

The term “isolated” refers to material which is substantially or essentially free from components that normally accompany it as found in its native state.

As used herein, “specific binding” refers to antibody binding to a predetermined antigen.

II. Liposome Composition and its Method of Preparation

In one aspect, the invention relates to a liposome composition prepared according to the method of the present invention. The following sections describe the liposome components, including the liposome lipids, lipid-linkers and therapeutic agents, preparation of liposomes bearing a targeting-ligand, and methods of using the liposomal composition for treatment of disorders.

A. Liposome Lipid Components

Liposomes suitable for use in the composition of the present invention include those composed primarily of vesicle-forming lipids. Such a vesicle-forming lipid is one which can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes.

The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose carbon chains have varying degrees of saturation can be obtained commercially or prepared according to published methods. Other suitable lipids include glycolipids, cerebrosides and sterols, such as cholesterol.

Cationic lipids are also suitable for use in the liposomes of the invention, where the cationic lipid can be included as a minor component of the lipid composition or as a major or sole component. Such cationic lipids typically have a lipophilic moiety, such as a sterol, an acyl or diacyl chain, and where the lipid has an overall net positive charge. Preferably, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethylammonium bromide (DORIE); N-[1-(2,3-dioleyloxy) propyl]-N,N,N-trimethylammonium chloride (DOTMA); 3 [N-(N′,N′-dimethylaminoethane) carbamoly] cholesterol (DC-Chol); and dimethyldioctadecylammonium (DDAB). The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

The vesicle-forming lipid can be selected to achieve a specified degree of fluidity or rigidity, to control the stability of the liposome in serum, to control the conditions effective for insertion of the targeting conjugate, as will be described, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

On the other hand, lipid fluidity is achieved by incorporation of a relatively fluid lipid, typically one having a lipid phase with a relatively low liquid to liquid-crystalline phase transition temperature, e.g., at or below room temperature.

The liposomes also include a vesicle-forming lipid covalently attached to a hydrophilic polymer, also referred to herein as a “lipopolymer”. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating.

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

A preferred hydrophilic polymer chain is polyethyleneglycol (PEG), preferably as a PEG chain having a molecular weight between 500-10,000 daltons, more preferably between 750-10,000 daltons, still more preferably between 750-5000 daltons. Methoxy or ethoxy-capped analogues of PEG are also preferred hydrophilic polymers, commercially available in a variety of polymer sizes, e.g., 120-20,000 Daltons.

Preparation of vesicle-forming lipids derivatized with hydrophilic polymers has been described, for example in U.S. Pat. No. 5,395,619. Preparation of liposomes including such derivatized lipids has also been described, where typically between 1-20 mole percent of such a derivatized lipid is included in the liposome formulation (see, for example, U.S. Pat. No. 5,013,556).

B. Ligand

The liposme composition also includes a ligand that is associated with or attached to the lipid-linker. The ligand is a protein or peptide. In one embodiment, the ligand is an antibody, antibody fragment Fab′, single chain Fv. In another embodiment, the ligand is a temperature sensitive protein or peptide. In yet another embodiment, the ligand is a temperature sentitive antibody, antibody fragment Fab′, single chain Fv.

In one embodiment, the liposome composition also includes an antibody that targets the lipid particles to a cell. In one embodiment, the antibody is one having affinity for a growth factor cell receptor. In a preferred embodiment, the antibody for use in the liposome composition described herein is described in WO 99/55367, the subject matter of which is incorporated herein by reference in its entirety.

C. Preparation of Lipid-Linker Conjugate

As described above, the ligand is covalently attached to the free distal end of a hydrophilic polymer chain, which is attached at its proximal end to a vesicle-forming lipid, after the lipid-linker is inserted into the liposome. There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Allen, T. M., et al., Biochemicia et Biophysica Acta, 1237:99-108 (1995); Zalipsky, S., Bioconjugate Chem., 4(4):296-299 (1993); Zalipsky, S., et al. FEBS Lett., 353:71-74 (1994); Zalipsky, S. et al., Bioconjugate Chemistry, 6(6):705-708 (1995); Zalipsky, S., in STEALTH LIPOSOMES (D. Lasic and F. Martin, Eds.) Chapter 9, CRC Press, Boca Raton, Fla. (1995)).

Generally, the PEG chains are functionalized to contain reactive groups suitable for coupling with, for example, sulfhydryls, amino groups, and aldehydes or ketones (typically derived from mild oxidation of carbohydrate portions of an antibody) present in a wide variety of ligands. Examples of such PEG-terminal reactive groups include maleimide (for reaction with sulfhydryl groups), N-hydroxysuccinimide (NHS) or NHS-carbonate ester (for reaction with primary amines), hydrazide or hydrazine (for reaction with aldehydes or ketones), iodoacetyl (preferentially reactive with sulfhydryl groups) and dithiopyridine (thiol-reactive). Synthetic reaction schemes for activating PEG with such groups are set forth in U.S. Pat. Nos. 5,631,018, 5,527,528, 5,395,619, and the relevant sections describing synthetic reaction procedures are expressly incorporated herein by reference.

An exemplary synthetic reaction scheme is described in U.S. Pat. No. 6,326,353. Briefly, polyethylene glycol (PEG) bis (amine) (Compound I) is reacted with 2-nitrobenzene sulfonyl chloride to generate the monoprotected product (Compound II). Compound II is reacted with carbonyl diimidazole in triethylamine (TEA) to form the imidazole carbamate of the mono 2-nitrobenzenesulfonamide (Compound III). Compound III is reacted with DSPE in TEA to form the derivatized PE lipid protected at one end with 2-nitrobenzyl sulfonyl chloride. The protecting group is removed by treatment with acid to give the DSPE-PEG product (Compound IX) having a terminal amine on the PEG chain. Reaction with maleic acid anhydride gives the corresponding maleamic product (Compound V), which on reaction with acetic anhydride gives the desired PE-PEG-maleimide product (Compound VI). The compound is reactive with sulfhydryl groups, for coupling a ligand after the lipid-polymer is inserted into a liposome through a thioether linkage (Compound VII).

It will be appreciated that any of the hydrophilic polymers recited above in combination with any of the vesicle-forming lipids recited above can be employed as modifying agents to prepare the lipid-polymer conjugate and suitable reaction sequences for any selected polymer can be determined by those of skill in the art.

D. Targeting-Liposome Preparation

As indicated above, the preparation of the targeting-liposome involves the preparation of the lipid-linker and the pre-formed liposome. The lipid-linker and the liposome are prepared as described above. In a preferred embodiment, micelles of PEG-lipid with a maleimide linker are prepared as the lipid-linker and a doxorubicin- encapsulated liposome derivatized with PEG is prepared as the liposome.

Next, the lipid-linker and the pre-formed liposome are combined. In a preferred embodiment, the lipid-linker and the pre-formed liposome are combined at a temperature that is sufficient to allow the lipid-linker to become incorporated into the pre-formed liposome. In a particularly preferred embodiment, the lipid-linker and the pre-formed liposome are combined at a temperature that is in the range from about 50 to about 70° C., and more preferably about 60° C. It should be understood, however, that the temperature at which the lipid-linker and pre-formed liposome are combined can vary and is dependent upon the lipid-linker and the pre-formed liposome. One of skill in the art will be able to identify the particular temperature.

In one embodiment of the present invention, the lipid-linker is incorporated with the pre-formed liposome for a period in the range of about 30 minutes to about 2 hours, and preferably about 1 hour. It should be understood, however, the amount of time that the lipid-linker and pre-formed liposome are combined can vary and is dependent upon the lipid-linker and the pre-formed liposome. One of skill in the art will be able to identify the particular amount of time.

The ligand is then combined with the lipid-linkers incorporated liposomes. The ligand is combined in a manner to allow the ligands to become associated or attached to the lipid-linkers incorporated into the pre-formed liposomes. In a preferred embodiment of the present invention, the ligand is combined with the lipid-linker incorporated liposomes at a temperature that is not adverse to the function of, or will denature, the ligand. In a particularly preferred embodiment of the present invention, the ligand is combined with the lipid-linker incorporated liposomes at a temperature that is lower than when the lipid-linkers are combined with the pre-formed liposomes. It should be understood, however, that the temperature at which the ligand and the lipid-linker incorporated liposome are combined can vary and is dependent upon the lipid-linker and the pre-formed liposome. One of skill in the art will be able to identify the particular temperature

In one embodiment of the present invention, the ligand and the lipid-linker incorporated liposome are combined for a period in the range of about 3 to about 6 hours, and preferably about 4 to about 5 hours. It should be understood, however, the amount of time that the lipid and the lipid-linker incorporated liposome are combined can vary and is dependent upon the ligand and the lipid-linker incorporated liposome. One of skill in the art will be able to identify the particular amount of time.

III. Methods of Use

The liposomes prepared according to the method of the present invention include a therapeutic or diagnostic agent in entrapped form. Entrapped is intended to include encapsulation of an agent in the aqueous core and aqueous spaces of liposomes as well as entrapment of an agent in the lipid bilayer(s) of the liposomes. Agents contemplated for use in the composition of the invention are widely varied, and examples of agents suitable for therapeutic and diagnostic applications are given below.

The dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. The dosage can be a one-time or a periodic dosage given at a selected interval of hours, days, or weeks.

Any route of administration is suitable, with intravenous and other parenteral modes being preferred.

In another aspect, the invention contemplates a combined treatment regimen, where the immunoliposome composition prepared according to the method of the present invention described above is administered in combination with a second agent. The second agent can be any therapeutic agent, including other drug compounds as well as biological agents, such as peptides, antibodies, and the like. The second agent can be administered simultaneously with or sequential to administration of the immunoliposomes, by the same or a different route of administration.

IV. Kits

The present invention also provides for kits for preparing the above-described targeting-liposomes. Such kits can be prepared from readily available materials and reagents, as described above. For example, such kits can comprise any one or more of the following materials: liposomes, proteins, lipid-linkers, namely hydrophilic polymers, hydrophilic polymers not derivatized with targeting moieties, and instructions. A wide variety of kits and components can be prepared according to the present invention, depending upon the intended user of the kit and the particular needs of the user.

### EXAMPLES

The following example further illustrates the invention described herein and is in no way intended to limit the scope of the invention.

### Example 1

The process for preparing a Fab′-conjugated STEALTH® liposomal doxorubicin (SL-DXR) immunoliposome by the method of the present invention is described. Antibody Fab′ is a very common ligand for immunoliposomes. In general, the lipid-linker, Mal-PEG-DSPE, was allowed to form micelles in an aqueous solution. The lipid anchor of Mal-PEG-DSPE was then incorporated into the lipid membrane of a pre-formed liposomes with encapsulated drugs, Doxil® liposome formulation, by incubation. The insertion efficiency reached up to 70-97% at high temperature (50-70° C.) for 1-4 hours. The high temperature was important, since Doxil® liposome formulation was composed of some high phase transition temperature lipids (around 55° cT). The desired ratio of the lipid-linkers per liposome was achieved in the range from about 10 to 50. After Mal-PEG-DSPE insertion, the free thiol groups of a Fab′ were reacted to the Mal-group on the liposome surface after incubation at room temperature for 1 hour. The conjugation efficiency was more than 95%.

In greater detail, Mal-PEG-DSPE (1.9 mg) was suspended in a MilliQ water (0.19 mL) to form a 10 mg/mL suspension. A solution of SL-DXR (e.g., 4.76 mg/mL of liposomal doxorubicin, 1.0 mL) was mixed with the suspension of Mal-PEG-DSPE (10 mg/mL, 81 μL), stirred at 62-65° C. for 1 h. Anti integrin αᵥβ₃ and αᵥβ₅ antibody Fab′ were obtained from Centocor, Inc. (See WO 2004/056323, the subject matter of which is incorporated by reference in its entirety). The Fab′ solution after reduction (1.1 mg/mL, 0.63 mL) was added to above Mal-PEG-DSPE inserted Doxil® suspensions. The reaction mixtures were stirred under Argon at room temperature (RT) for 1 h. The un-reacted Mal- of conjugation products was quenched by cysteine (10 mg/mL, 33 μL) at RT for 1 h. The conjugation yield was determined by taking out 0.1 mL and mixing with IAC (10 L) for SDS-PAGE gel analysis. The (Fab′)₂ and unconjugated Fab′ were removed by Sepharose 4B SEC column (25 cm×1 cm) using 10 mM histidine-0.9% sodium chloride as the eluate. The fractions were determined by O.D. at 280 nm and 480 nm. The product fractions were combined, concentrated by centrifuge at 3200 rpm, and a sample was pulled out (20 μL) for SDS PAGE-gel analysis. The doxorubicin concentration was determined by O.D. at 480 nm. The particle size was measured by light scattering machine, and the insertion efficiency was checked by HPLC.

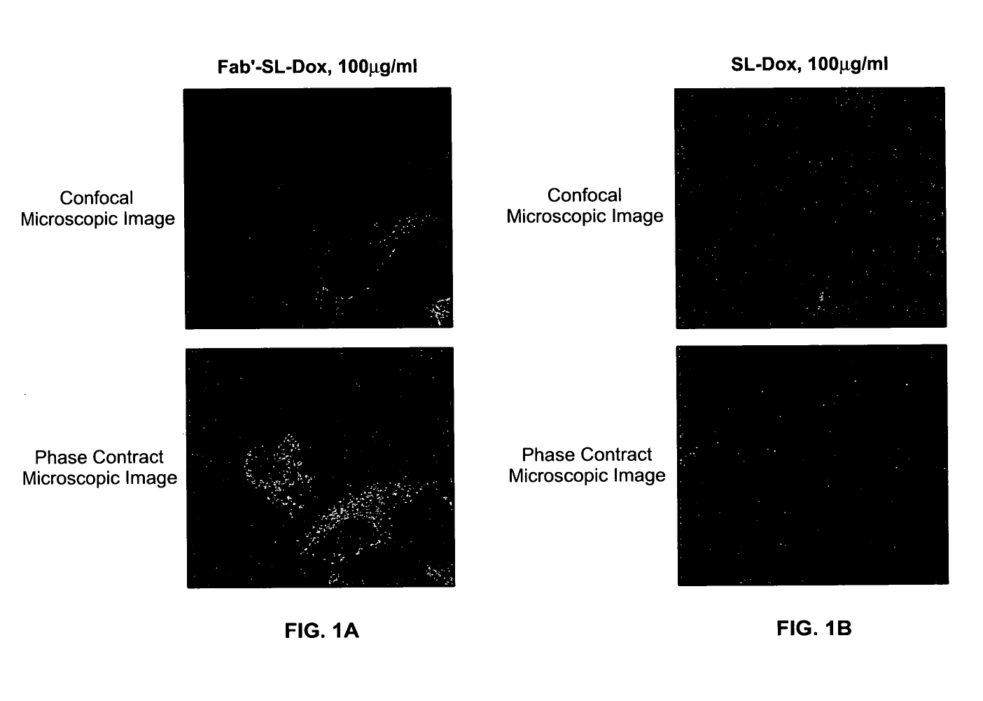

The internalization was evaluated by confocal microscopy as follows. A375-S2 human melanoma cell line which expresses integrin αᵥβ₃ and αᵥβ₅ receptors were used in this study. Cells grew overnight and then were treated with Fab′-conjugated SL-DXR or SL-DXR without Fab′-conjugation at the concentration of 100 μg/ml doxorubicin in serum free media for 15 minutes, respectively. After washing the cells were resuspended with media containing 10% serum and incubated at 37° C. for 1.5 hours. Internalization of Fab′-conjugated liposomes was observed by confocal microscope [FIG. 1 (a)], in contrast, there was not significant internalization of the liposomes without Fab′-conjugation [FIG. 1 (b)].

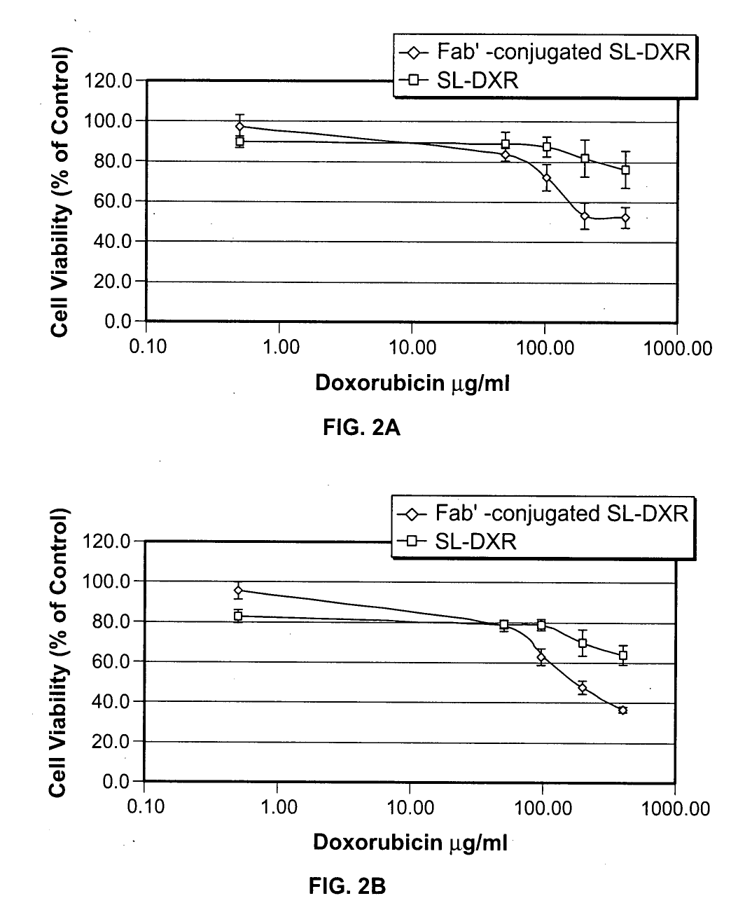

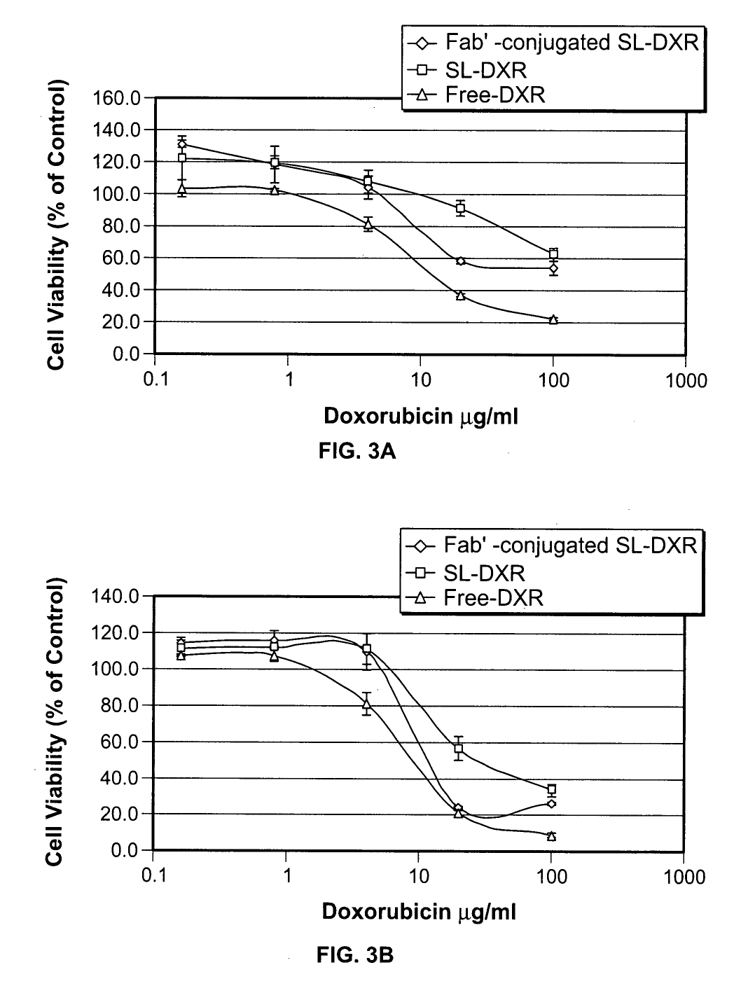

In vitro tumor cell inhibition study was as follows. A375-S2 human melanoma tumor cells were trypsinized from the flask to make single cell suspension. The cell suspension (2 million cells in 1 ml per test tube, about 10 ml depending on experiment scale) was incubated at 37° C. for 2 hours in cell culture medium with FBS and was shaken mildly. Cells were spun down by 2010 rps for 5 minutes at 4° C., and the supernatant was discarded. Next was added 0.1 ml of warm (37° C.) Fab′-SL-DXR or SL-DXR treatment solution which was diluted with cold cell culture medium without FBS to reach various treatment concentrations in each test tube, and the warm cell culture medium without FBS was added to the cells as a control. The cells were taped to mix. The cells were incubated at and kept at 37° C. for 15 min, and were shaken mildly in room temperature. The treatment was stopped by adding 1 ml of cell culture medium without FBS at 37° C. The cells were spun down at room temperature and washed with 1 ml of warm (37° C.) cell culture medium without FBS. The cells were shook vigorously for 10 minutes at room temperature. The spin and wash process was repeated twice. The cells were spun down at room temperature, and the supernatant was discarded. The cells were resuspended with 1 ml of cell culture medium with FBS (37° C.). The cells were counted, and 2,000 cells were seeded into each well of a 96 well plate. Triplets were set up for each treatment point. The cells were incubated at 37° C. for 6 days and cell inhibition rate (% of cell viability) was determined. The cytotoxicity difference between targeted liposomes and non-targeted liposomes was due to the dominant effect of the specific binding of the antibody Fab′ (FIGS. 2A-B). The difference of cytotoxicity between targeted liposomes and non-targeted liposome due to the specific binding and internalization is indicated in FIGS. 3A-B. In this experiment the condition was similar to the condition in FIG. 1, except the treatment was at 37° C., and the processes were at RT or 37° C. also.

Although the invention has been described with respect to particular embodiments, it will be apparent to those skilled in the art that various changes and modifications can be made without departing from the invention.

It is claimed:

1. A method for preparing liposomal compositions, the method comprising the steps of:
combining a lipid-linker with a pre-formed liposome having an entrapped therapeutic agent at a temperature sufficient to allow insertion of the lipid-linker into the pre-formed liposome; and
conjugating ligands to the lipid-linkers at a lower temperature so that the ligands are not adversely denatured.

2. The method of claim 1, wherein the lipid-linker is combined with the pre-formed liposome at a temperature sufficient to meet the phase transition temperature of the lipids in the pre-formed liposome.

3. The method of claim 1, wherein the lipid-linker is combined with the pre-formed liposome at a temperature in the range from about 50-70° C.

4. The method of claim 1, wherein the lipid-linker is combined with the pre-formed liposome at a temperature in the range from about 50-70° C. and the ligands are conjugated to the lipid-linkers at about room temperature.

5. The method of claim 1, wherein the lipid-linker is combined with the pre-formed liposome at temperatures in the range from about 60° C. and the ligands are conjugated to the lipid-linkers covalently on the surface of the pre-formed liposome at about room temperature.

6. The method of claim 1, wherein the lipid-linker is combined with the pre-formed liposome at temperatures in the range from about 60° C. for about 1 hour, and the ligands are conjugated to the lipid-linkers covalently on the surface of the pre-formed liposome at about room temperature.

7. The method of claim 1, wherein the lipid-linker is combined with the pre-formed liposomes in an efficiency of up to about 97% and the ligands are conjugated to the lipid-linkers covalently on the surface of the pre-formed liposome at about room temperature.

8. The method of claim 1, wherein the lipid-linker is combined with the pre-formed liposomes in an efficiency of about 90 to about 97% and the ligands are conjugated to the lipid-linkers covalently on the surface of the pre-formed liposome at about room temperature.

9. The method of claim 1, wherein the ligand is sensitive to high temperatures.

10. The method of claim 1, wherein the ligand is sensitive to high temperatures and high pH conditions.

11. The method of claim 1, wherein the ligand is for a HER2 receptor.

12. The method of claim 1, wherein the ligand is for a growth factor receptor.

13. The method of claim 12, wherein the ligand is for epidermal growth factor recptor.

14. The method of claim 1, whrerein the lipid-linker comprises a hydrophilic polymer poly(ethylene glycol).

15. The method of claim 1, wherein the liposomal compositions have a size between about 50-100 nm.

16. The method of claim 1, wherein the pre-formed liposomes have an anthracycline as the entrapped therapeutic agent.

17. The method of claim 1, wherein the pre-formed liposome is composed of at least about 20 mole percent of a vesicle-forming lipid and at least about 1 mole percent of a vesicle-forming lipid derivatized with a hydrophilic polymer, said polymer being distributed on both sides of the liposomes' bilayer membrane.

18. The method of claim 1, wherein the lipid-linker is a vesicle-forming lipid derivatized with a hydrophilic polymer that has a reactive end.

19. The method of claiml8, wherein the lipid-linker is Mal-PEG-DSPE.

20. A product prepared according to the process for preparing a liposomal composition, the process comprising the steps of:
combining a lipid-linker with a pre-formed liposome having an entrapped therapeutic agent at a temperature sufficient allow insertion of the lipid-linker into the pre-formed liposome; and
conjugating ligands to the lipid-linkers at a lower temperature so that the ligands are not adversely denatured.
